(19) United States
(12) Patent Application Publication
(10) Pub. No.: US 2023/0355342 A1
(43) Pub. Date: Nov. 9, 2023

(54) Surgical Drapes

(71) Applicant: ART, LIMITED, Grand Cayman (KY)

(72) Inventors: Takayuki Akahoshi, Tokyo (JP); Ravi Nallakrishnan, Willowbrook, IL (US)

(21) Appl. No.: 18/027,199

(22) PCT Filed: Sep. 30, 2021
(86) PCT No.: PCT/US2021/052856

§ 371 Date: Mar. 20, 2023

Related U.S. Application Data
(60) Provisional application No. 63/087,092, filed on Oct. 2, 2020.

(51) Int. Cl. A61B 46/20

(52) U.S. Cl. CPC A61B 46/20; A61B 2046/205

(57) ABSTRACT

A surgical drape (20A) is provided for use while performing surgical procedures on one or both eyes of a patient, or other surgical procedures. The surgical drape includes a base sheet (24A), defining a surgical aperture (28A) having the form of an elongate slot sized to accommodate both eyes of the patient. The drape (20A) includes an attachment zone (32A) formed on the lower surface of the base sheet (24A) to fix at least a portion of the base sheet (24A) with respect to the patient. The attachment zone (32A) permitting attachment of a first portion of the base sheet lower surface to a second portion of the base sheet lower surface to vary the length of the surgical aperture (28A) to accommodate a pupillary distance of the patient.

### PRIORITY

This application claims priority of U.S. Provisional Patent Application No. 63/087,092, filed Oct. 2, 2020, the entire contents of which is incorporated herein by reference.

### FIELD OF THE INVENTION

The present invention relates generally to the field of surgery including ophthalmic surgery, and more particularly, to a surgical drape for use during surgery of the human eye.

### BACKGROUND OF THE INVENTION

When the eye becomes aged, diseased, or injured it may be necessary to remove the natural lens of the eye. Such removal is common for cataract surgery, in which a lens that has become clouded is removed. The removal of the natural lens of the eye may result in the loss or alteration of focused vision of a patient. Eyeglasses, contact lenses, or the implantation of an artificial lens may be necessary to restore the vision of the patient.

During the removal of the natural lens of the eye, a physician will typically make one or more small incisions in the eye and insert a vibratory needle into the incision. The needle may be vibrated at ultrasonic speeds in torsional, longitudinal, elliptical, or blended modes in order to dismember the lens in a process known as phacoemulsification. The needle typically contains one or more aspiration passages for removing the broken lens particles from the eye. Lasers may also be used to dismember the lens.

This surgical process is typically performed with an ophthalmic drape being placed over the eye of the patient. The ophthalmic drape has one aperture for being placed over the eye upon which surgery will occur. The drape has an aperture for insertion of the surgical equipment and may have an adhesive to secure the drape to the patient's skin. A topical or local anesthetic is applied to the eye. However, general anesthesia may be required for some patients who cannot remain still for the surgical procedure or for some patients who may suffer from claustrophobia. The obstructed vision of the eye which is not being operated upon may further exacerbate such claustrophobia. General anesthetic can add substantial expense to the surgical operation and may further present risks to the health of the patient, when compared to local anesthetic. A bilateral, two aperture, type drape for addressing claustrophobia is described in PCT publication no. WO/2020/191120, which is incorporated herein by reference in its entirety.

The inventors have further found that patients have a pupillary distance that varies considerably from patient to patient presenting difficulty in manufacturing and applying a standard bilateral (two aperture) type drape across a variety of patients.

The inventors have found that the conventional surgical drape for ophthalmic surgery, which has a rectangular shaped pouch to collect fluid during surgery, may suffer from instability as fluid collects in the pouch. Specifically, the fluid within such pouches may shift from corner to corner, causing the pouch to move or pull the drape relative to the patient.

The inventors of the present invention have determined that, in at least some applications, it would be desirable to provide an improved drape that is adjustable across a wide variety of patients.

The inventors of the present invention have further determined that, for at least some applications, it may be desirable to provide such an improved drape that can be easily applied to the surgical site of the patient with improved stability.

The inventors of the present invention have also determined that it would be desirable to provide, at least for some applications, an improved drape that can allow the user to selectively protect the areas not being operated upon while mitigating or minimizing the likelihood of the patient experiencing claustrophobia.

The inventors of the present invention have also discovered that it would be desirable to provide, at least for some applications, an improved drape that can be manufactured and/or assembled at a relatively low cost, and can accommodate manufacture of the actuator by means of efficient, high-quality, large-volume techniques.

The inventors of the present invention have discovered how to provide a drape that includes novel, advantageous features not heretofore taught or contemplated by the prior art, and which can accommodate designs having one or more of the above-discussed benefits or features.

### SUMMARY OF THE INVENTION

The present invention is directed to a surgical drape with a wide range of potential applications.

In accordance with one broad form of the present invention, a surgical drape is provided, which is particularly useful for ophthalmic surgery, including a bilateral procedure. The drape has a base sheet of flexible material. The base sheet has an upper surface, a lower surface for contacting a patient, and at least one edge therebetween. The base sheet includes a surgical aperture in the form of an elongate slot formed through the base sheet, with the surgical aperture having a length to accommodate a first and second eye of the patient. An attachment zone is formed on the lower surface of the base sheet at least partially surrounds the surgical aperture. The attachment zone permits attachment of the base sheet lower surface to the patient to fix at least a portion of the base sheet with respect to the patient. The attachment zone further is configured to permit attachment of a first portion of the base sheet lower surface to a second portion of the base sheet lower surface to vary the length of the surgical aperture to accommodate a pupillary distance of the patient

In accordance with one preferred form of the present invention, the base sheet includes a raised crease formed adjacent, above and below, the surgical aperture at a location where a first portion of the base sheet lower surface is attached to a second portion of the base sheet lower surface.

In accordance with another broad form of the present invention, a surgical drape is provided, which is particularly useful for ophthalmic surgery, including a bilateral procedure. The drape has a base sheet of flexible material. The base sheet has an upper surface, a lower surface for contacting a patient, and at least one edge therebetween. The base sheet includes at least one surgical aperture formed through the base sheet, with the surgical aperture having a length and positionable proximal to a first eye of the patient. An attachment zone is formed on the lower surface of the base sheet at least partially surrounds the surgical aperture. The attachment zone permits attachment of the base sheet lower surface to the patient to fix at least a portion of the base sheet with respect to the patient. The drape includes at least one pouch positioned in proximity to the at least one surgical aperture. The pouch includes an opening and a pair of tapered sides that extend toward one another in a direction away from the at least one surgical aperture to minimize shifting of the drape due to fluid collection in the pouch.

In one preferred form of the present invention, the pouch has a pentagonal shape and the pair of tapered sides terminate at a point. In another preferred form of the present invention, the pouch includes a pressure-sensitive adhesive region for permitting the pouch to be removably attached to the upper surface of the base sheet.

In accordance with yet another broad form of the present invention, a surgical drape is provided, which is particularly useful for ophthalmic surgery, including a SMILE or refractive surgical procedure. The drape has a base sheet of flexible material. The base sheet has an upper surface, a lower surface for contacting a patient, and at least one edge therebetween. The base sheet includes at least one surgical aperture formed through the base sheet, with the surgical aperture having a length and positionable proximal to a first eye of the patient. An attachment zone is formed on the lower surface of the base sheet at least partially surrounds the surgical aperture. The attachment zone permits attachment of the base sheet lower surface to the patient to fix at least a portion of the base sheet with respect to the patient. The drape includes a speculum connected to the base sheet and positioned in proximity to the at least one aperture. The speculum has a first and a second arm that are resiliently movable relative to one another about a pivot. The pivot is constrained to the top or upper surface of the base sheet at a lateral location adjacent the at least one aperture.

According to one preferred form of the invention, the pivot is non-adjustably constrained to the base sheet.

According to another preferred form of the invention, the pivot is adjustably constrained to the base sheet.

In accordance with still another broad form of the present invention, a surgical drape is provided, which is particularly useful for ophthalmic surgery, including a bilateral procedure. The drape has a base sheet of flexible material. The base sheet has an upper surface, a lower surface for contacting a patient, and at least one edge therebetween. The base sheet includes at least one surgical aperture formed through the base sheet, with the surgical aperture having a length and positionable proximal to a first eye of the patient. An attachment zone is formed on the lower surface of the base sheet at least partially surrounds the surgical aperture. The attachment zone permits attachment of the base sheet lower surface to the patient to fix at least a portion of the base sheet with respect to the patient. The drape includes at least one protective patch for positioning at and substantially covering at least a portion of the surgical aperture to protect the eye of the patient. The protective patch has the form of a polymeric sheet having pressure-sensitive adhesive applied to one surface thereof, and defines an elongated slit through which the surgical procedure is performed. The protective patch including at least one indicium located at a center of the elongated slit to facilitate placement of the patch on the eye of the patient.

In one preferred form of the present invention, the pressure sensitive adhesive of the protective patch is provided in a region surrounding the slit. In yet another preferred form of the present invention, the at least one indicium is a central line that extends transverse to the length of the slit. In another preferred form of the present invention, the protective patch is generally rectangular and defines an asymmetric tab portion extending transverse to, and located on one side of, the slit.

In accordance with still another broad form of the present invention, a surgical drape is provided, which is particularly useful for ophthalmic surgery, including a bilateral procedure. The drape has a base sheet of flexible material. The base sheet has an upper surface, a lower surface for contacting a patient, and at least one edge therebetween. The base sheet includes at least one surgical aperture formed through the base sheet, with the surgical aperture having a length and positionable proximal to a first eye of the patient. An attachment zone is formed on the lower surface of the base sheet at least partially surrounds the surgical aperture. The attachment zone permits attachment of the base sheet lower surface to the patient to fix at least a portion of the base sheet with respect to the patient. The drape includes at least one substantially transparent shield attached to the base sheet and extending across at least a portion of the surgical aperture.

In one preferred form of the present invention, the substantially transparent shield includes a pressure-sensitive adhesive and is removably attached to the base sheet. According to another preferred form the present invention, the substantially transparent shield has a rectangular shape. In still another form of the present invention, the substantially transparent shield has the form of a pair of substantially transparent shields affixed to the upper surface of the base sheet, and each one of the pair is spaced laterally, one from the other, in a non-contacting relationship.

Another feature of the present invention is to provide a low-cost, disposable surgical drape for the reduction and/or minimization of claustrophobia in a patient.

In another aspect of the present invention, a folded surgical drape is provided to facilitate positioning of the drape on a patient, and subsequent spreading of the drape over the patient.

Other features and advantages of the present invention will be better understood with reference to the accompanying figures and detailed description.

### BRIEF DESCRIPTION OF THE DRAWINGS

FIG. 1 is a diagrammatic, top plan view of a first embodiment of a disposable surgical drape embodying the principles of the present invention shown in an unfolded configuration;

FIG. 2 is a more detailed, diagrammatic, top plan view of the drape of FIG. 1, and FIG. 2 shows the preferred dimensions of the drape in millimeters;

FIG. 3 is a more detailed, diagrammatic, bottom plan view of the drape of FIG. 1, and FIG. 3 shows the preferred dimensions of the drape in millimeters, and FIG. 3 shows a removable pouch that is resting on the top (opposite) surface of the base sheet;

FIG. 4 is a diagrammatic, top plan view of a protective patch which can be selectively secured to the base sheet of the disposable surgical drape according to the present invention;

FIG. 5 is a diagrammatic, bottom plan view of the protective patch shown in FIG. 4 (with the slit omitted), illustrating removal of a protective liner or paper layer from the pressure-sensitive adhesive provided on the bottom side of the protective patch;

FIG. 6 is a diagrammatic, top plan view of the variation of pupillary distance of two different patients;

FIG. 7 is a diagrammatic, top plan view showing a conventional unilateral (single aperture) drape used on the two different patients of FIG. 6;

FIG. 8 is a diagrammatic, top plan view showing a conventional bilateral (dual aperture) drape used on the two different patients of FIG. 6, and FIG. 8 shows the difficulties in sizing one drape to fit patients with a different pupillary distance;

FIG. 9 is a diagrammatic, top plan view showing a conventional bilateral (dual aperture) drape used on the two different patients of FIG. 6, and FIG. 9 shows the difficulties in placing a protective patch on patients having a different pupillary distance;

FIG. 10 is a diagrammatic, top plan view showing the drape of FIG. 1 used on the two different patients of FIG. 6, and FIG. 10 shows the ability of the drape to be effectively used on patients having a different pupillary distance;

FIGS. 11 and 12 are perspective, enlarged diagrammatic views showing the drape of FIG. 10 used on the two different patients;

FIGS. 13 and 14 are perspective, enlarged diagrammatic views showing the drape of FIG. 10 used on the two different patients;

FIG. 15 is a diagrammatic, top plan view of a removable pouch of the drape of FIG. 1 (at the far left) compared to two prior art pouches (at the right);

FIG. 16 is an isometric view, from above of a second embodiment of a disposable surgical drape embodying the principles of the present invention shown in an unfolded configuration;

FIG. 17 is an enlarged, fragmentary view of the top surface of the drape shown in FIG. 16;

FIG. 18 is an isometric view, from below of the bottom surface of the drape shown in FIG. 16;

FIG. 19 is an enlarged, fragmentary view of the bottom surface of the drape shown in FIG. 18;

FIG. 20 is a photographic, top plan view of the drape shown in FIG. 16, however FIG. 20 shows a variation of the attachment means of the speculum to the top surface of the drape;

FIG. 21 is a photographic, perspective view from above of a third embodiment of a disposable surgical drape embodying the principles of the present invention shown in an unfolded configuration;

FIG. 22 is a photographic, perspective view from above of the drape of FIG. 21, and FIG. 22 shows a user of the drape beginning to remove a protective, transparent shield from above a surgical aperture;

FIG. 23 is a photographic, perspective view from above of the drape of FIG. 21, and FIG. 23 shows a user of the drape continuing to remove a protective, transparent shield to expose a surgical aperture;

FIG. 24 is a photographic, perspective view from above of the drape of FIG. 21, and FIG. 24 shows the drape in a folded configuration with two pouches attached to the upper surface of the base sheet and with the transparent shields removed;

FIG. 24 is a photographic, perspective view from above of the upper surface of the drape of FIG. 21, and FIG. 24 shows the drape in a folded configuration with two pouches attached to the upper surface of the base sheet and with the transparent shields removed;

FIG. 25 is a photographic, perspective view from above of the upper surface of the drape of FIG. 24, and FIG. 25 shows the drape in a partially unfolded configuration;

FIG. 26 is a photographic, perspective view from above of the upper surface of the drape of FIG. 25, and FIG. 26 shows the drape in an unfolded configuration;

FIG. 27 is a photographic, perspective view from above of the upper surface of the drape of FIG. 26;

FIG. 28 is a photographic, perspective view from below of the lower surface of the drape of FIG. 25, and FIG. 28 shows the backing paper partially removed to reveal the attachment zone adhesive; and

FIG. 29 is a photographic, perspective view from below of the lower surface of the drape of FIG. 25, and FIG. 29 shows the backing paper completely removed to reveal the attachment zone adhesive.

### DETAILED DESCRIPTION OF THE PREFERRED EMBODIMENTS

While the present invention is susceptible of embodiment in various forms, there is shown in the drawings and will hereinafter be described presently preferred embodiments, with the understanding that the present disclosure is to be considered an exemplification of the invention, and is not intended to limit the invention to the specific embodiments illustrated.

The present disposable surgical drapes are particularly suited for use in connection with ophthalmic surgical procedures, in particular a bilateral procedure. However, features of the surgical drapes permit them to be advantageously used in other procedures, including central venous catheterization (CVC), other catheterization procedures, anesthesia procedures, and other surgical procedures, as will be described.

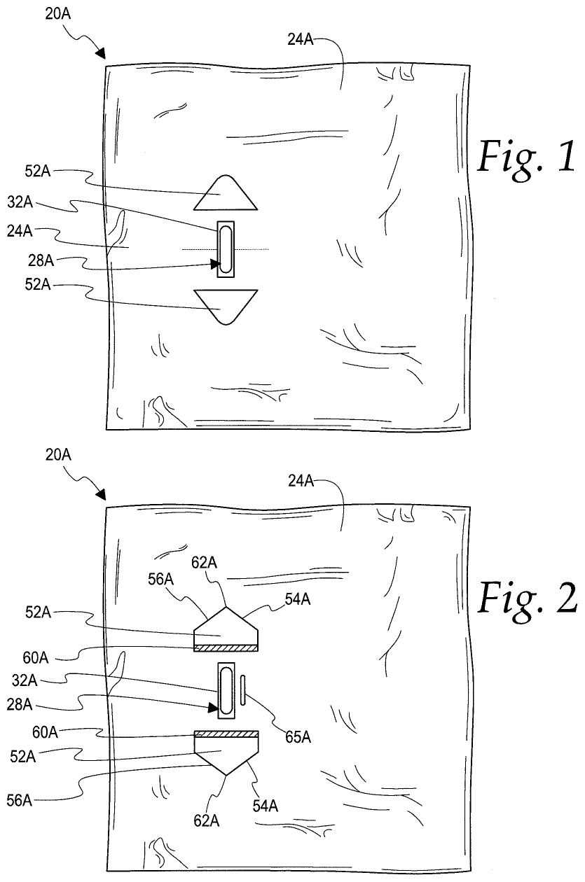

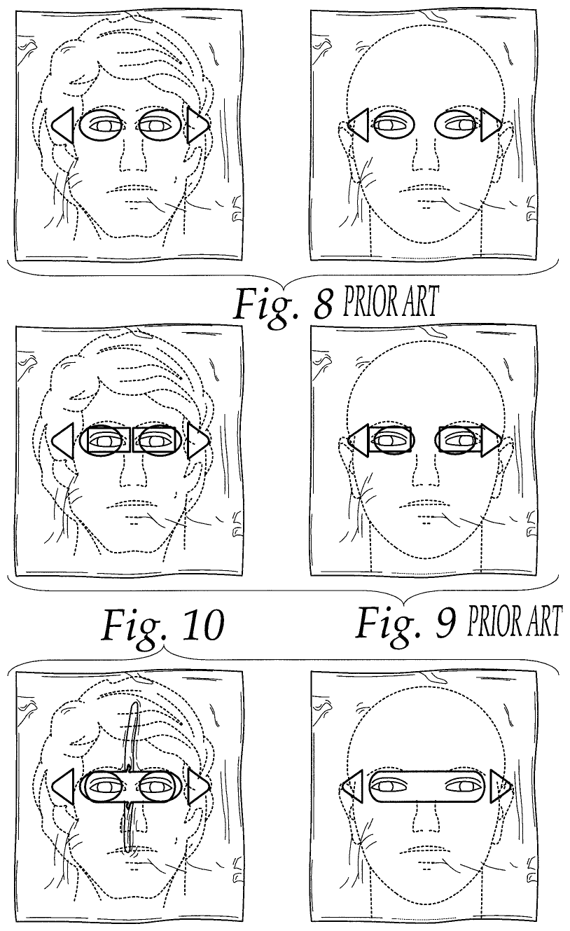

FIGS. 1-4, and 9-13 illustrate a first embodiment of a disposable surgical drape 20A, which is particularly useful for ophthalmic procedures, embodying the principles of the present invention. The first illustrated embodiment of the drape 20A is formed from a generally rectangular base sheet 24A made from a thin, flexible material. Base sheet 24A can be made from tissue paper, textiles, fluid-permeable and non-fluid permeable polymers, and/or composites thereof. Preferably, the base sheet 24A is constructed from a light, water resistant paper that will be disposed of after a single use. In another presently preferred form, the base sheet 24A comprises a composite non-woven SMS (spunbond-meltblown-spunbond) fabric. The drape 20A need not have a generally rectangular base sheet 24A, but may have any of a variety of shapes, such as polygonal, circular, oval, or irregular shapes to conform to the area of the patient undergoing the surgical procedure.

The base sheet 24A has an upper surface (visible in FIGS. 1 and 2), a lower surface (visible in FIG. 3), and an edge therebetween. The lower surface is positionable upon and in contact with the face of a patient undergoing an ophthalmic surgery.

As seen in FIG. 2, the base sheet 24A defines a single surgical aperture 28A extending through the base sheet 24A for being respectively positioned over both of the eyes of a patient during ophthalmic surgery. The aperture 28A has the form of an elongate slot or racetrack shape, which functions to accommodate a pupillary distance of the patient, as will be discussed in greater detail hereinafter. The aperture 28A may be initially formed in the base sheet 24A, cut from the base sheet 24A, or subsequently created by tearing or rupturing of the base sheet 24A along a weakened or perforated area. The aperture 28A can also be formed by incision or piercing of the base sheet 24A by a surgical instrument. Preferably, the aperture 28A is large enough to provide the patient with an unobstructed view of his or her surroundings to minimize the likelihood of claustrophobia. The aperture 28A preferably extends parallel to, and offset from, an adjacent top edge of the base sheet 24A which would be placed over or at the top of the head of the patient.

As can be seen in FIG. 3, a bottom plan view of the drape 20A showing the lower surface of the base sheet 24A thereof, drape 20A further has an attachment zone 32A, preferably comprising a pressure-sensitive adhesive at least partially surrounding the aperture 28A so that the base sheet 24A can be removably affixed and secured to the face of the patient during the surgical procedure or procedures. In the preferred form, the attachment zone extends completely about the aperture 28A. A protective layer or backing sheet (not illustrated) is preferably removably secured to the pressure-sensitive adhesive of the attachment zone 32A to maintain the adhesive in a fresh condition prior to use.

Notably, the provision of the single surgical apertures 28A in the form of an elongate slot facilitates a surgical procedure on a variety of patients having different pupillary distances, which allows for a standard drape to be used across a variety of patients. The slot desirably minimizes any claustrophobia that the patient might experience that is associated with unilateral (single aperture) prior art drapes, since one eye can remain uncovered while the surgical procedure is performed on the other eye. The provision of such stereoscopic vision to the patient can provide a low-cost alternative to general anesthetic and can reduce the movement of an anxious, claustrophobic patient. In some forms, the surgical aperture 28A may be provided with a ruler or other indicia indicating a predetermined pupillary distance or distances to assist the user in quickly and repeatably adjusting the length of the surgical aperture 28A during use on a variety of patients, as will be discussed in greater detail. While the surgical aperture 28A has the form of a racetrack shape, with rounded ends and a rectangular middle section, it will be understood that the slot may have the form of an oval, square, etc.

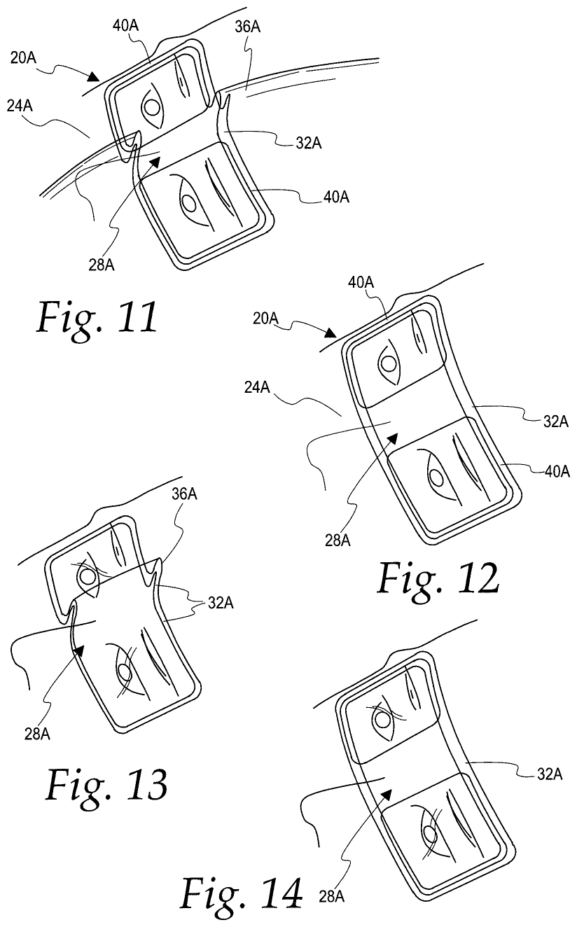

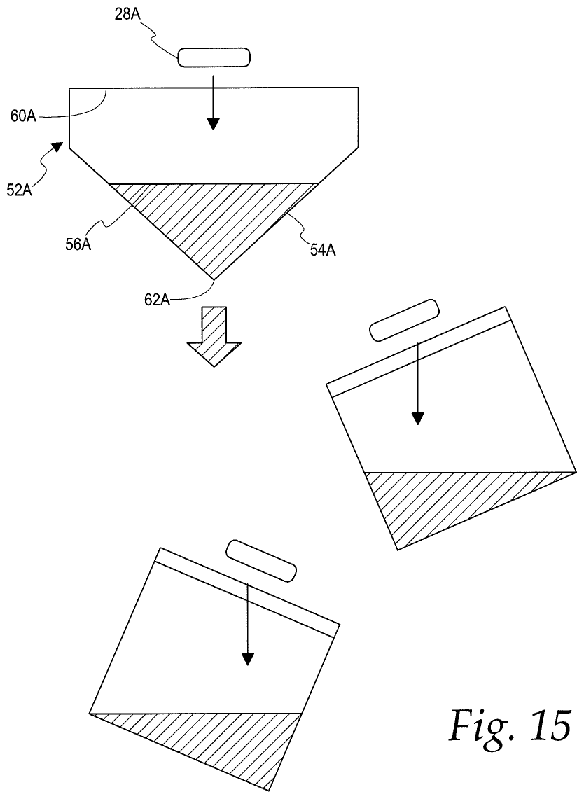

As can be seen in FIGS. 11 and 13, the attachment zone 32A permits the attachment of a first portion of the base sheet lower surface to a second portion of the base sheet lower surface to vary the length of the surgical aperture 28A to accommodate a pupillary distance of the patient. A fold or raised crease 36A, where the first and second portions of the attachment zone 32A is adhered to itself, may be formed in the base sheet 24A when the surgical aperture 28A is shortened to accommodate a patient with a relatively short pupillary distance, while not fold or crease 36A may be formed when the drape 20A is applied to a patient having a relative large pupillary distance.

With reference to FIGS. 4, 5, 11 and 12, the surgical drape 20A further includes at least one protective patch 40A for positioning at and covering an eye of the patient that is exposed within the single surgical aperture 28A to protect the eye of the patient when the drape 20A is attached to the patient via the attachment zone adhesive. Preferably, the drape 20A includes a pair of the protective patches 40A, each of which can be used once for the procedure to be performed on each of the eyes of the patient.

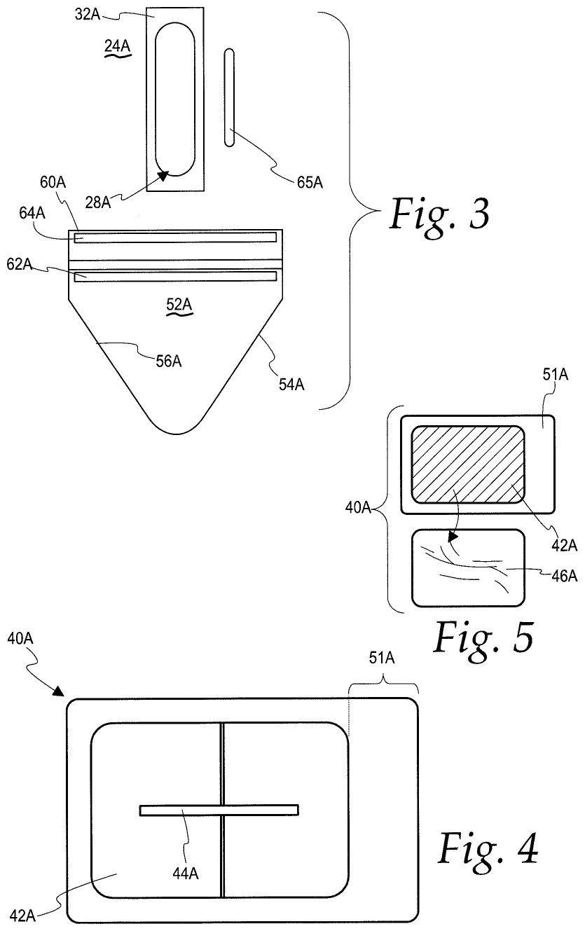

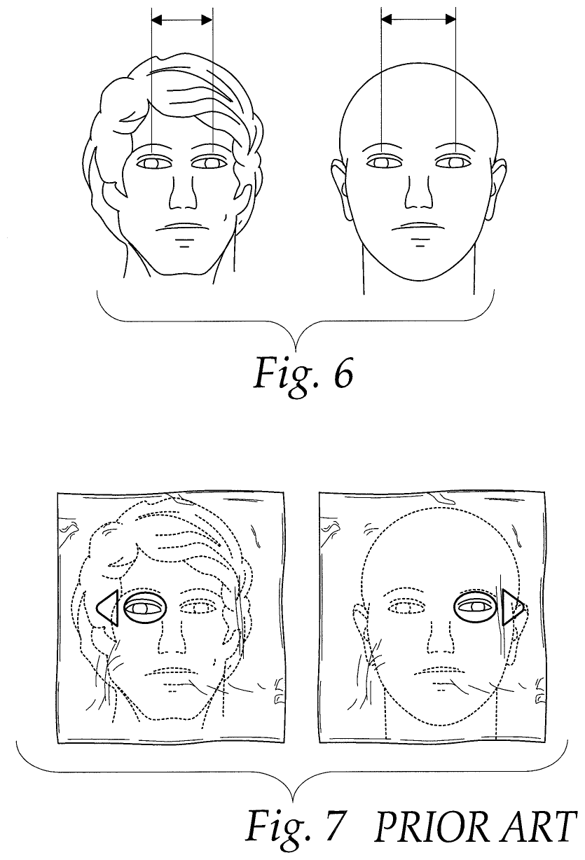

Each protective patch 40A has the form of a polymeric sheet having a pressure-sensitive adhesive area or region 42A (visible in FIGS. 4 and 5) applied to one surface surrounding an elongated slit 44A through which the surgical procedure is performed. The pressure-sensitive adhesive on each of protective patches 40A is preferably provided on an area or region fully surrounding the slit 44A defined by the protective patch and is further provided with a removable protective layer or backing paper 46A to maintain the pressure-sensitive adhesive 42A in a fresh condition prior to use. The patches 40A have a generally rectangular shape and define an asymmetric tab portion 51A extending transverse to, and located on one side of, the slit 44A for more efficient removal of the paper 46A from the adhesive 42A. The top surface of the patch 40A includes at least one indicium or indicia 49A located at a center of the elongated slit 44A to assist in the centration of the patch 40A on the eyelids of the patient. In one preferred form of the invention, the indicium 49A has the form of a central line extending transverse or normal to the length of the slit 44A. In some forms, the patches 40A may have non-rectangular shapes, and may be oval, square, or other polygonal or irregular shapes.

As can further be seen in FIG. 2, the drape 20A further preferably includes at least one, and more preferably a pair of, detachable pouches or compartments 52A attached to the upper surface of base sheet 24A. Each pouch 52A is preferably located proximal to one of the opposing ends of the elongate single surgical aperture 28A, with an opening or mouth 60A of each pouch facing toward the respective end of the surgical aperture 28A. The mouth 60A includes a wire element 62A that may be adjusted to maintain the mouth 60A in an open or closed position and an adhesive region or strip 64A located on the lower surface of the pouch 52A extending parallel to the mouth 60A. Each pouch 52A has a pentagonal shape with a base defined by the mouth 60A and a pair of opposing, tapered sides 54A, 56A that may join or terminate at a point 62A. The tapered sides 54A, 56A extend toward the point 62A in a direction away from the surgical aperture 28A. The inventors have found that the tapering design of the pouches 52A may facilitate stability and enhanced collection of fluid compared to the pouches of the prior art designs. The pouches 52A are designed to catch any overflow of balanced salt solution that may not be aspirated into the surgical devices used in the surgical procedure. Typically, the balanced salt solution will be applied to the eye undergoing surgery as an irrigation solution to maintain pressure within the eye. The balanced salt solution is then typically aspirated through one or more lumens for sanitary disposal. Excess balanced salt solution that is not aspirated into such lumens may collect on the base sheet 24A. The pouches 52A help to eliminate or at least control the accumulation of balanced salt solution. Alternatively, the pouches 52A can retain various surgical tools, equipment, and/or supplies when the drape 20A is draped over and attached to the face of the patient.

The pouches 52A may be integrally formed with the drape via heat welding, adhesive, or stitching to the upper surface of the base sheet 24A. Preferably, the pouches 52A are removable to reduce the weight of the base sheet 24A on the face of the patient to reduce impediments to breathing which could increase a sense of claustrophobia. In one preferred form of the invention, the pouch 52A would be applied just prior to the operation on a particular adjacent eye of the patient, then the pouch 52A would be removed just after the operation.

With reference to FIG. 2, the drape 20A is provided with a wire element 65A located below the surgical aperture 28A where the patient's nose would be located when the drape 20A is affixed to the patient. The wire 65A provides stability to the drape 20A and further allows the user to modify the fit of the drape 20A with the patient's face.

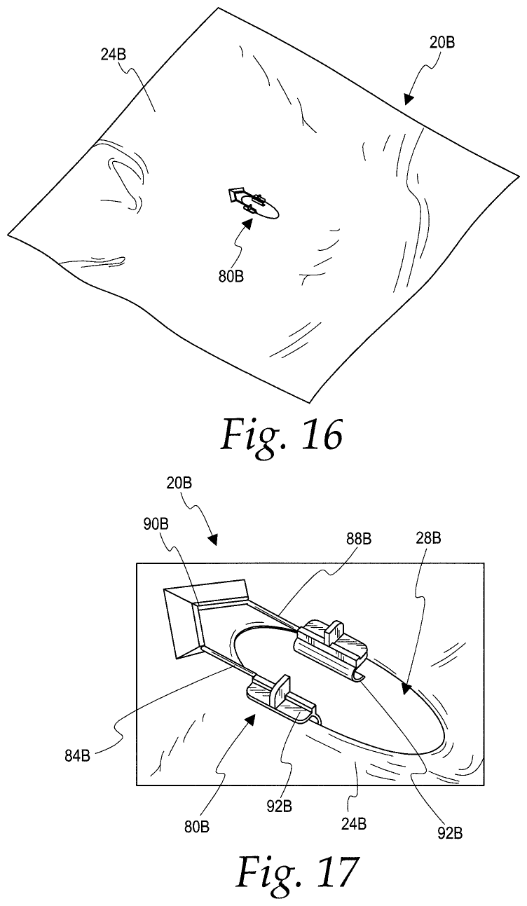

FIGS. 16-20 illustrate a second embodiment of a surgical drape 20B, which is particularly useful for ophthalmic procedures, embodying the principles of the present invention. Like elements between the first illustrated embodiment of the drape 20A and the second illustrated embodiment of the drape 20B are designated with the same numeral (the first embodiment having an “A” suffix and the second embodiment having a “B” suffix). The second illustrated embodiment of the drape 20B shares several of the same basic elements as the first illustrated embodiment of the drape 20A. For example, the drape 20B is formed from a generally rectangular base sheet 24B made from a thin, flexible material. Base sheet 24B can be made from tissue paper, textiles, fluid-permeable and non-fluid permeable polymers, and/or composites thereof. Preferably, the base sheet 24B is constructed from a light, water resistant paper that will be disposed of after a single use. In another presently preferred form, the base sheet 24B comprises a composite non-woven SMS (spunbond-meltblown-spunbond) fabric. The drape 20B need not have a generally rectangular base sheet 24B, but may have any of a variety of shapes, such as polygonal, circular, oval, or irregular shapes to conform to the area of the patient undergoing the surgical procedure.

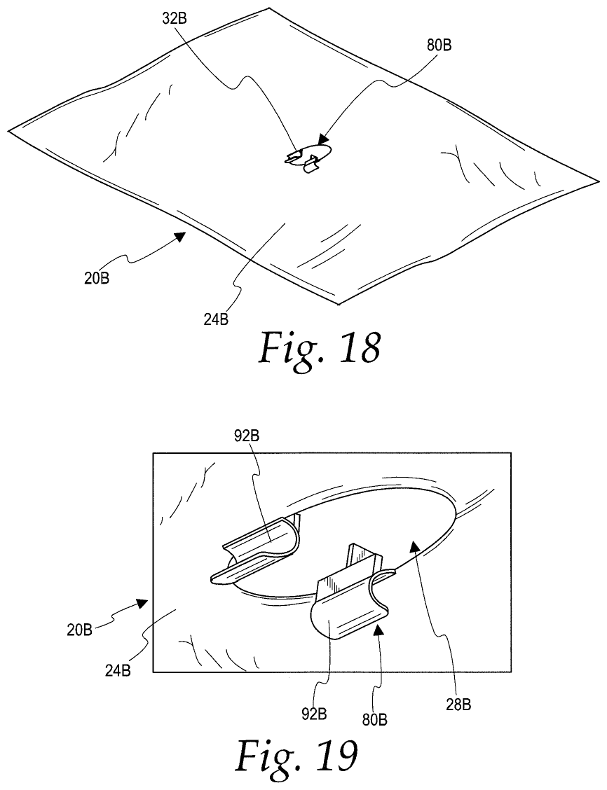

The base sheet 24B has an upper surface (visible in FIG. 16), a lower surface (visible in FIG. 18), and an edge therebetween. The lower surface is positionable upon and in contact with the face of a patient undergoing an ophthalmic surgery.

As seen in FIG. 17, the base sheet 24B defines at least one surgical aperture 28B extending through the base sheet 24B for being respectively positioned over an eye of a patient during ophthalmic surgery. The aperture 28B has the form of an oval shape, which functions to overlie an eye of the patient, as will be discussed in greater detail hereinafter. The aperture 28B may be initially formed in the base sheet 24B, cut from the base sheet 24B, or subsequently created by tearing or rupturing of the base sheet 24B along a weakened or perforated area. The aperture 28B can also be formed by incision or piercing of the base sheet 24B by a surgical instrument. Preferably, the aperture 28B is large enough to provide the patient with an unobstructed view of his or her surroundings to minimize the likelihood of claustrophobia. The aperture 28B preferably extends parallel to, and offset from, an adjacent top edge of the base sheet 24B which would be placed over or at the top of the head of the patient.

The bottom the lower surface of the base sheet 24B of the drape 20B may be provided with or without an attachment zone 32B, preferably comprising a pressure-sensitive adhesive at least partially surrounding the aperture 28B so that the base sheet 24B can be removably affixed and secured to the face of the patient during the surgical procedure or procedures. In the preferred form, the attachment zone extends completely about the aperture 28B. A protective layer or backing sheet (not illustrated) is preferably removably secured to the pressure-sensitive adhesive of the attachment zone 32B to maintain the adhesive in a fresh condition prior to use.

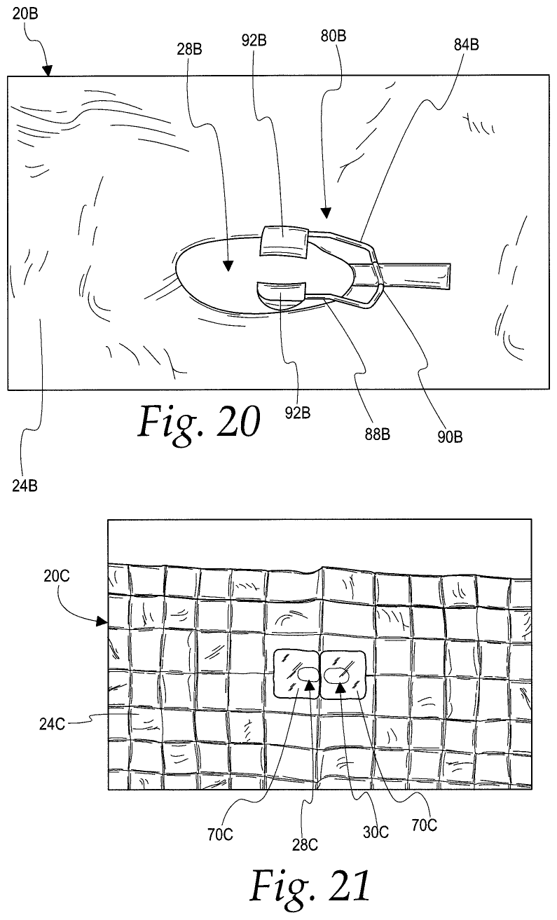

With reference to FIGS. 17 and 19, the drape 20B includes a speculum 80B connected to the top or upper surface of the base sheet 24B and positioned in proximity to the surgical aperture 28B. The speculum 80B includes first and second arms 84B, 88B that are movable relative to one another about a pivot 90B. Each arm terminates in a blade 92B cantilevered within the aperture 28B and sized and shaped to engage the eyelids of a patient. The pivot 90B may be fixedly secured to the top of the base sheet 24B (FIG. 17) or may be adjustably constrained to the top surface of the base sheet 24B (FIG. 20) at a lateral location adjacent to the aperture 28B. In one form, the attachment of the pivot 90B with the speculum 80B may be substantially fixed by a tape, adhesive, or weld extending in a V-shaped path to allow for substantially no appreciable lateral movement of the pivot 90B toward or away from the aperture 28B. In an alternative form, the pivot 90B may be axially constrained by laterally adjustable toward or away from the aperture 28B by way of a loop of material such as tape constraining the pivot 90B.

Notably, the provision of the speculum 80B adjacent to the aperture 28B facilitates the maintenance of the orientation of the speculum 80B to lock it in place relative to the drape 20B. The inventors believe that the construction of the drape 20B having a speculum 80B attached at the pivot 90B may be especially suitable for use in small-incision lenticule extraction refractive surgery.

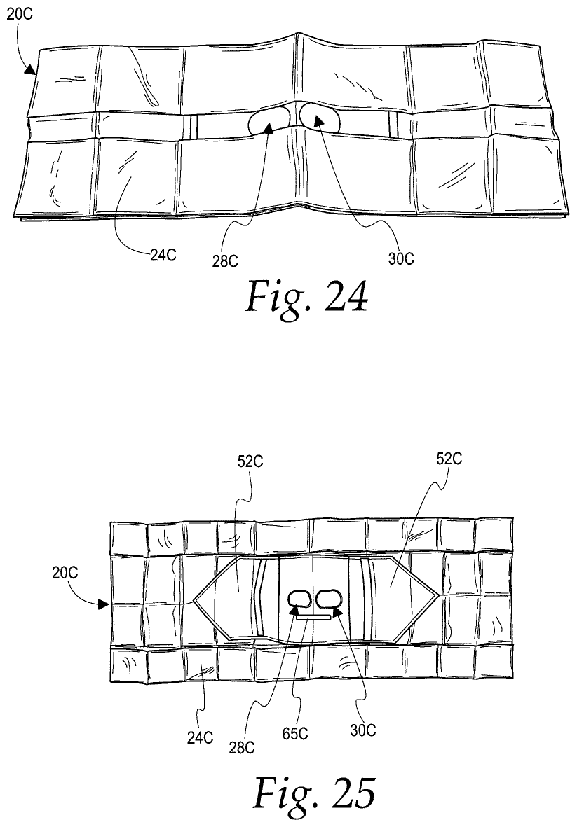

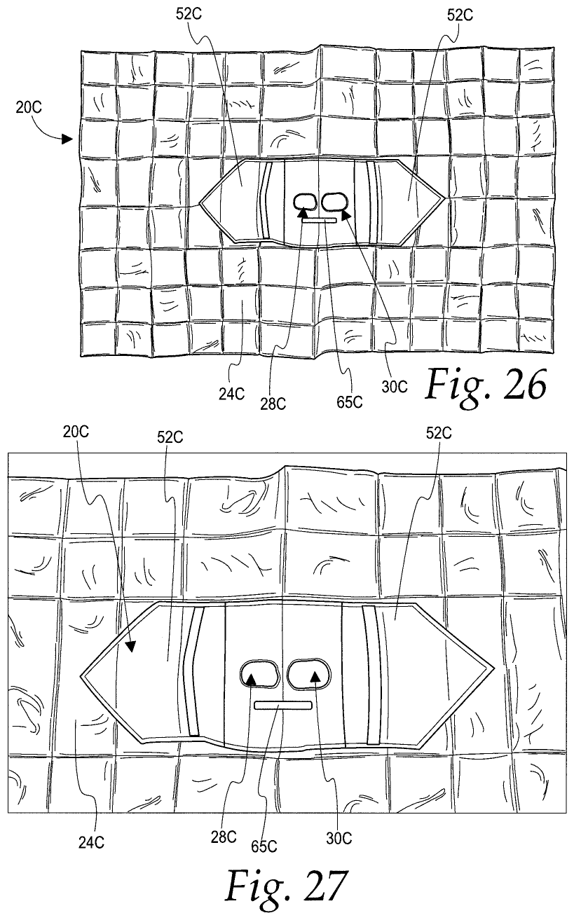

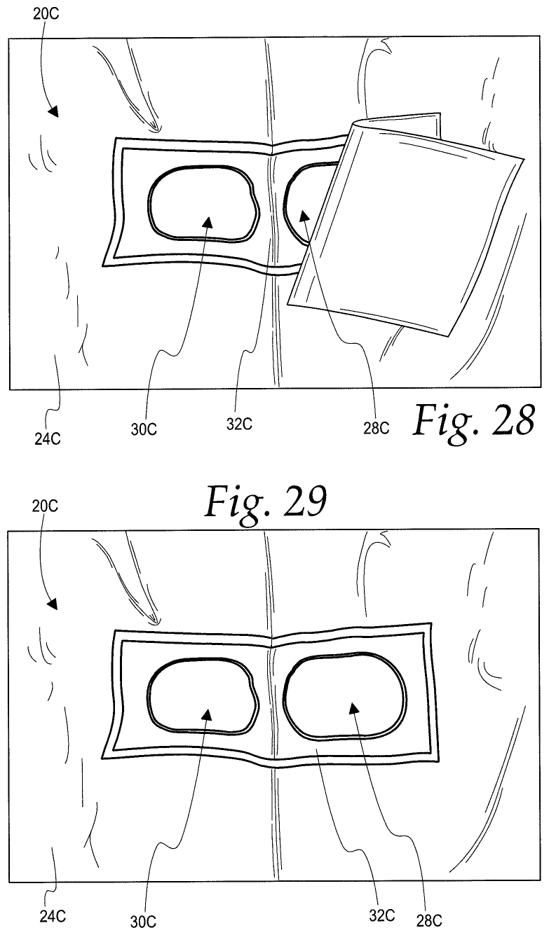

FIGS. 21-29 illustrate a third embodiment of a surgical drape 20C, which is particularly useful for ophthalmic procedures, embodying the principles of the present invention. Like elements between the first illustrated embodiment of the drape 20A and the third illustrated embodiment of the drape 20C are designated with the same numeral (the first embodiment having an “A” suffix and the third embodiment having a “C” suffix). The third illustrated embodiment of the drape 20C shares several of the same basic elements as the first illustrated embodiment of the drape 20C. For example, the drape 20C is formed from a generally rectangular base sheet 24C made from a thin, flexible material. Base sheet 24C can be made from tissue paper, textiles, fluid-permeable and non-fluid permeable polymers, and/or composites thereof. Preferably, the base sheet 24C is constructed from a light, water resistant paper that will be disposed of after a single use. In another presently preferred form, the base sheet 24C comprises a composite non-woven SMS (spunbond-meltblown-spunbond) fabric. The drape 20C need not have a generally rectangular base sheet 24C, but may have any of a variety of shapes, such as polygonal, circular, oval, or irregular shapes to conform to the area of the patient undergoing the surgical procedure.

The base sheet 24C has an upper surface (visible in FIG. 21), a lower surface (visible in FIG. 29), and an edge therebetween. The lower surface is positionable upon and in contact with the face of a patient undergoing an ophthalmic surgery.

As can be seen in FIG. 21, the base sheet 24C defines first and second surgical apertures 28C, 30C extending through the base sheet 24C for being respectively positioned over the eyes of a patient during ophthalmic surgery. The apertures 28C, 30C are typically initially formed in the base sheet 24C, cut from the base sheet 24C, or are subsequently created by tearing or rupturing of the base sheet 24C along a weakened or perforated area. The apertures 28C, 30C can also be formed by incision or piercing of the base sheet 24C by a surgical instrument. The apertures 28C, 30C may be in the shape of a circle, oval, or elongate slot shape. Preferably, the apertures 28C, 30C are large enough to provide the patient with an unobstructed view of his or her surroundings to minimize the likelihood of claustrophobia.

The lower surface of the base sheet 24C may be provided with an attachment zone (32C in FIG. 29), as previously discussed with respect to the other illustrated embodiments of the invention, preferably comprising a pressure-sensitive adhesive at least partially surrounding the apertures 28C, 30C so that the base sheet 24C can be removably affixed and secured to the face of the patient during the surgical procedure or procedures. In the preferred form, the attachment zone 32C extends completely about the apertures 28C, 30C. A protective layer may be removably secured to the pressure-sensitive adhesive of the attachment zone to maintain the adhesive in a fresh condition prior to use. While the attachment zone 32C may fully surround the surgical apertures 28C, 30C, the attachment zone may alternatively be provided only partially surrounding the surgical apertures 28C, 30C, or may be located elsewhere on the base sheet lower surface.

The provision of first and second surgical apertures 28C, 30C facilitates the surgical procedure, and desirably minimizes any claustrophobia that the patient might experience, since one eye can remain uncovered while the surgical procedure is performed on the other eye. The inventor has found that providing a pair of surgical apertures, used in sequence such as during a bilateral procedure, can desirably minimize the anxiety of claustrophobic patients because the vision of the second eye is not obscured by the base sheet 24C during the operation. The provision of such stereoscopic vision to the patient can provide a low-cost alternative to general anesthetic and can reduce the movement of an anxious, claustrophobic patient.

However, the inventors have found that the exposed eye not undergoing a surgical procedure may be at risk for damage during operation of the other exposed eye. Accordingly, the drape 20C includes a pair of independent shields 70C removably attached or affixed to the top surface of the base sheet 24C. Each shield 70C is substantially transparent to a patient (high transmittance of visible light of greater than about 75%) and extends across one of the surgical apertures 28C, 30C. For example, the shields may be a formed from transparent vinyl, PET, acrylic, polycarbonate, polystyrene, or other suitable polymers. The shields 70C are preferably formed from sufficiently rigid polymer to protect against impacts and are fluid resistant. Each transparent shield 70C includes a pressure-sensitive adhesive on its lower side, which is removably attached to the base sheet 24C. As shown in FIGS. 21-23, each shield 70C is rectangular and is independently affixed to the base sheet 24C.

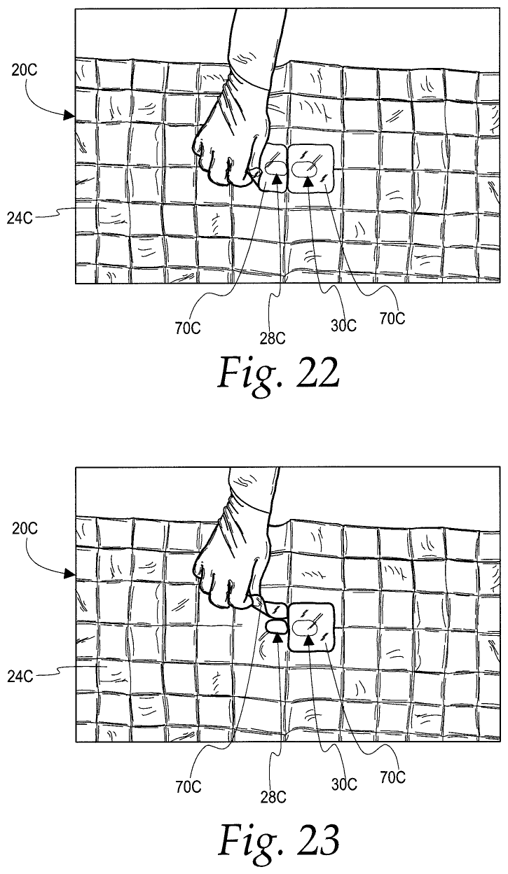

It is contemplated that the drape 20C would be manufactured with a shield 70C across each surgical apertures 28C, 30C and the user of the drape 20C would remove a first shield 70C to expose a first eye undergoing a surgical procedure. For example, upon removal of the first shield 70C to expose the aperture 28C placed over top of a first eye undergoing a surgical procedure (as shown in FIGS. 22 and 23), the surgeon would affix a surgical slit patch, such as the patch 40A in FIG. 4, across the exposed aperture 28C. Upon completion of the surgery of the first eye, the patch 40A would be removed along and the first shield 70C would be reapplied over the aperture 28C. Next, the surgeon would remove the second shield 70C to expose the aperture 30C to expose a second eye for undergoing a surgical procedure. In this manner, each that is not undergoing surgery is protected and provided with a largely unobstructed view to reduce claustrophobia.

The drape 20C may further be provided with one or more detachable pouches or compartments 52C in FIG. 26, attached to the upper surface of base sheet 24C. Each pouch 52C is located proximal to a respective one of the surgical apertures 28C, 30C, with an opening of each pouch facing toward the respective surgical aperture to catch any overflow of balanced salt solution that may not be aspirated into the surgical devices used in the surgical procedure. A wire or bridge 65C is further provided for accommodating the nose of the patient in an axial direction below the apertures 28C, 30C.

One method of use of the ophthalmic drape 20A for covering a patient, such as for bilateral eye surgery, will now be described. In this regard, it will be noted that the drape 20A is preferably manufactured in a folded fashion (not illustrated) such that lateral margins or edges of the base sheet 24A are folded so that the surgical apertures 28A remains uncovered by the lateral margins. This facilitates placement of the drape 20A on a patient, and securement of the attachment zone 32A of the drape to the face of the patient so the desired surgical procedure can be performed.

After removal of protective layer 42A from the attachment zone 32A to expose the pressure-sensitive adhesive, the surgical drape 20A is placed on the face of the patient and secured with the adhesive of the attachment zone. The lateral margins of the base sheet 24A can then be unfolded, with the drape 20A now in position with the lower surface of the base sheet 24A attached to the patient. The patient is thus covered in the desired fashion for performing the surgical procedure. The surgical aperture 28A are respectively positioned over the eyes of the patient.

With reference to FIGS. 11 and 12, depending on the length of the patient's pupillary distance, the attachment zone 32A permits the attachment of a first portion of the base sheet lower surface to a second portion of the base sheet lower surface to vary the length of the surgical aperture 28A to accommodate a pupillary distance of the patient. A fold or raised crease 36A, where the first and second portions of the attachment zone 32A is adhered to itself, may be formed in the base sheet 24A when the surgical aperture 28A is shortened to accommodate a patient with a relatively short pupillary distance, while not fold or crease 36A may be formed when the drape 20A is applied to a patient having a relative large pupillary distance.

As shown in FIGS. 11 and 12, one or more protective one protective patches 40A and/or shields 70C are positioned over the surgical aperture 28A to protect one or both eyes of the patient, depending on the nature of the procedure. Specifically, the protective layer 46A of the protective patch 40A is removed to expose the pressure-sensitive adhesive 42A. The protective patch 40A is applied over the surgical aperture 28A with the protective patch 40A thus removably secured to the base sheet 24A with the pressure-sensitive adhesive. The surgical procedure is them performed through the slit 44A in the protective patch 40A, while the other eye may be covered by a shield 70C (FIG. 21).

As noted, in the preferred form, a pair of detachable pouches 52A are provided on the base sheet 24A, positioned in proximity to each end of the elongate surgical apertures 28A to catch any overflow of balanced salt solution that may not be aspirated into the surgical devices used in the surgical procedure.

The presently preferred method of use of the drape 20A contemplates that after the surgical procedure is performed on the first eye of the patient, the protective patch 40A above, and the detachable pouch 52A adjacent, the first eye are removed from the base sheet 24A and discarded after the surgery on the first eye so as to relieve the weight of the drape 20A on the patient. Preferably, the adhesive of the protective patch 40A is selected such that it does not carry substantially any of the material of the drape base sheet 24A upon removal such that the drape base sheet 24A remains in place upon removal of the patch 40A. Thereafter, a second, fresh protective patch 40A is applied to the base sheet 24A over top of the other end of the aperture 28A over the other eye of the patient for a subsequent surgical procedure. A shield 70C may then be applied over the first eye to protect against inadvertent impact or contamination.

It is contemplated that the above-described embodiment may include a nose clip or wire 65A, or like spacing element, in order to provide height and clearance between the drape and the patient to facilitate convenient and comfortable breathing by the patient.

In the foregoing description, the present surgical drape has been described in connection with ophthalmic surgical procedures, but it is to be appreciated that the present surgical drape, including an attachment zone provided by a minimally-adhesive film, can be advantageously employed for other types of surgical procedures.

The present disposable surgical drape can be provided with an average size of 110 cm×110 cm, with surgical aperture 28A appropriately sized, such as approximately 14 cm×4 cm.

In summary, the present surgical drape provides a variety of desirable benefits. The drape is cost-effective, and can be used very efficiently to cover and protect any patient, regardless of pupillary distance.

Because both eyes of the patient can remain open, the present surgical drape desirably minimizes any claustrophobia the patient may experience. Having both eyes uncovered permits the uncovered eye to lead and fix the position of the eye on which the surgery is being performed.

Use of the present surgical drape is also recommended for routine use for various procedures in anesthesia and the intensive care unit

The illustrated preferred embodiments are included herein for descriptive purposes only and are not to be interpreted as limiting in any way of the broadest concepts of the present invention.

1. A surgical drape, comprising:
a base sheet of flexible material, said base sheet having an upper surface, and a lower surface for contacting a patient;
a surgical aperture formed through said base sheet, said surgical aperture having the form of an elongate slot and having a length sized to accommodate a first eye and a second eye of the patient,
an attachment zone formed on said base sheet lower surface, said attachment zone being located proximal to said surgical aperture, said attachment zone at least partially surrounding said at least one surgical aperture, said attachment zone permitting attachment of said base sheet lower surface to the patient to fix at least a portion of said base sheet with respect to the patient, and said attachment zone permitting attachment of a first portion of said base sheet lower surface to a second portion of said base sheet lower surface to vary said length of said surgical aperture to accommodate a pupillary distance of the patient.

2. The disposable surgical drape of claim 1, further comprising a raised crease formed adjacent said surgical aperture at a location where a first portion of said base sheet lower surface is attached to a second portion of said base sheet lower surface.

3. A surgical drape, comprising:
a base sheet of flexible material, said base sheet having an upper surface, and a lower surface for contacting a patient;
at least one surgical aperture formed through said base sheet, said surgical aperture having a length, said surgical aperture positionable proximal to a first eye of the patient;
an attachment zone formed on said base sheet lower surface, said attachment zone being located proximal to said at least one surgical aperture, said attachment zone at least partially surrounding said at least one surgical aperture, said attachment zone permitting attachment of said base sheet lower surface to the patient to fix at least a portion of said base sheet with respect to the patient; and
at least one pouch positioned in proximity to said at least one surgical aperture, said at least one pouch having an opening and a pair of tapered sides that extend toward one another in a direction away from said at least one surgical aperture.

4. The surgical drape of claim 3, wherein said at least one pouch has a pentagonal shape and said pair of tapered sides terminate at a point.

5. The surgical drape of claim 3, wherein said at least one pouch includes a pressure-sensitive adhesive region for being removably attached to said base sheet.

6. A surgical drape, comprising:
a base sheet of flexible material, said base sheet having an upper surface, and a lower surface for contacting a patient;
at least one surgical aperture formed through said base sheet, said at least one surgical aperture having a length, said surgical aperture positionable proximal to a first eye of the patient;
an attachment zone formed on said base sheet lower surface, said attachment zone being located proximal to said at least one surgical aperture, said attachment zone at least partially surrounding said at least one surgical aperture, said attachment zone permitting attachment of said base sheet lower surface to the patient to fix at least a portion of said base sheet with respect to the patient; and
a speculum connected to said base sheet and positioned in proximity to said at least one aperture, said speculum having first and second arms movable relative to one another about a pivot, said pivot being adjustably constrained to said top surface of said base sheet at a lateral location adjacent said at least one aperture.

7. A surgical drape, comprising:
a base sheet of flexible material, said base sheet having an upper surface, and a lower surface for contacting a patient;
a surgical aperture formed through said base sheet, said surgical aperture having a length, said surgical aperture positionable proximal to a first eye of the patient;
an attachment zone formed on said base sheet lower surface, said attachment zone being located proximal to said surgical aperture, said attachment zone at least partially surrounding said surgical aperture said attachment zone permitting attachment of said base sheet lower surface to the patient to fix at least a portion of said base sheet with respect to the patient; and
at least one protective patch for positioning at said surgical aperture to protect an eye of the patient,
said at least one protective patch comprising a polymeric sheet having pressure-sensitive adhesive applied to one surface thereof, and defining an elongated slit, whereby after positioning of said base sheet on said patient with said surgical aperture aligned with the eye of the patient, said protective patch can be applied over said surgical aperture and removably secured to said base sheet with said pressure-sensitive adhesive, said protective patch including at least one indicium located at a center of said elongated slit.

8. The disposable surgical drape of claim 7, wherein
said pressure sensitive adhesive of said protective patch is provided in a region surrounding said slit.

9. The disposable surgical drape of claim 7, wherein
said at least one indicium is a central line extending transverse to the length of said slit.

10. The disposable surgical drape of claim 7, wherein
said protective patch is generally rectangular and defines an asymmetric tab portion extending transverse to, and located on one side of, said slit.

11. A surgical drape, comprising:
a base sheet of flexible material, said base sheet having an upper surface, and a lower surface for contacting a patient;
at least one surgical aperture formed through said base sheet, said surgical aperture having a length, said surgical aperture positionable proximal to a first eye of the patient;
an attachment zone formed on said base sheet lower surface, said attachment zone being located proximal to said at least one surgical aperture, said attachment zone at least partially surrounding said at least one surgical aperture, said attachment zone permitting attachment of said base sheet lower surface to the patient to fix at least a portion of said base sheet with respect to the patient; and
at least one substantially transparent shield attached to said base sheet and extending across said at least one surgical aperture.

12. The disposable surgical drape of claim 11, wherein said at least one substantially transparent shield includes a pressure-sensitive adhesive and is removably attached to said base sheet.

13. The disposable surgical drape of claim 11, wherein said at least one substantially transparent shield has a rectangular shape.

14. The disposable surgical drape of claim 11, wherein said at least one substantially transparent shield comprises a pair of substantially transparent shields affixed to said upper surface of said base sheet, said pair being spaced laterally from one another in a non-contacting relationship.

15.-28. (canceled)
